(19) United States
(12) Patent Application Publication
(10) Pub. No.: US 2006/0205751 A1
(43) Pub. Date: Sep. 14, 2006

(54) Novel compounds

(75) Inventors: Dennis Lee, King of Prussia, PA (US); Joseph P. Marino, King of Prussia, PA (US); Yongdong Zhao, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SmithKline Beecham Corporation, Philadelphia, PA (US)

(21) Appl. No.: 10/564,451

(22) PCT Filed: Jul. 15, 2004
(86) PCT No.: PCT/US04/22706

§ 371 Date: Jan. 12, 2006

Related U.S. Application Data
(60) Provisional application No. 60/487,492, filed on Jul. 15, 2003.

(51) Int. Cl. A61K 31/4196; A61K 31/41; A61K 31/4192; A61K 31/4178; A61K 31/405; A61K 31/381; A61K 31/343

(52) U.S. Cl. 514/281; 514/381; 514/359; 514/383; 514/397; 514/443; 514/469; 514/419

(57) ABSTRACT

Compounds of the formula (I) are disclosed which are large conductance calcium activated potassium channel openers (BK channels openers) and are useful in the treatment of urinary tract disorders: 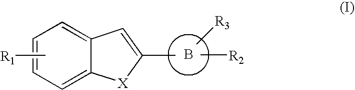 or a pharmaceutically acceptable salt thereof.

### FIELD OF THE INVENTION

The present invention relates to pharmaceutically active compounds, to pharmaceutical compositions containing them, and to their use In the treatment of disorders associated with potassium channel activation. Such disorders include cerebral infarction, dimentia, Alzheimer's disease, Parkinson's disease, suprasacral spinalcord disease, central nervous system disorders, hypertension, stroke, angina, congestive heart failure, subarachnoid hemorrhage, pollakiuria, urinary incontinence, urge incontinence, overactive bladder, diseases associated with detrusor instability, irritable bladder, irritable bowel syndrome, cystitis, urethritis, kidney stone ailments, diverticuli or outflow obstruction, and brochial asthma, pain, inflammatory pain, neuropathic pain and chronic obstructive pulmonary disease (COPD).

### BACKGROUND OF THE INVENTION

Potassium is the most abundant intracellular cation and is very important in maintaining physiological homeostasis. Potassium channels are present in almost all vertebrate cells and the potassium influx through these channels is indispensable for maintaining hyperpolarized resting membrane potential.

Large conductance calcium activated potassium channels (also BK channels or maxi-K channels) are expressed in neurons, cardiac and smooth muscle cells. Maxi-K channels have been thought to play a pivotal role in regulating voltage-dependant calcium influx because these channels are activated by both the increase intracellular calcium concentration and membrane depolarization. Increase in the intracellular calcium concentration mediates many processes such as release of neurotransmitters, contraction of smooth muscles, cell growth and death. Actually, the opening of maxi-K channels causes strong membrane hyperpolarization and thereby inhibits these calcium-induced responses. Accordingly, by inhibiting various depolarization-mediated physiological responses, a substance having an activity of opening maxi-K channels is expected to have potential for the treatment of cerebral infarction, dimentia, Alzheimer's disease, Parkinson's disease, suprasacral spinalcord disease, central nervous system disorders, hypertension, stroke, angina, congestive heart failure, subarachnoid hemorrhage, pollakiuria, urinary incontinence, urge incontinence, overactive bladder, diseases associated with detrusor instability, irritable bladder, irritable bowel syndrome, cystitis, urethritis, kidney stone ailments, diverticuli or outflow obstruction, and brochial asthma, pain, inflammatory pain, neuropathic pain and chronic obstructive pulmonary disease (COPD).

### DETAILED DESCRIPTION OF THE INVENTION

This invention comprises a method of treating or inhibiting disorders associated with the activation of large conductance calcium activated potassium channels, which comprises administering to a subject in need thereof an effective amount of a compound according to formula (I): 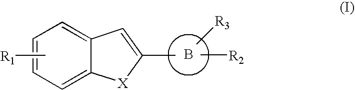
wherein:
R₁ is absent or represents up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, trifluoromethyl, cyano, COₐ, CO₂Rₐ, SO₃H, (C₁₋₆)alkyl-CO₂—(C₁₋₆)alkyl, CONRₐR_{b}, and NRₐR_{b};
X is NRₐ, O, or S;
B is aryl or heterocycle;
R₂ is absent or represents is up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, cyano, CORₐ, CO₂Rₐ, SO₃H, (C₁₋₆)alkyl-CO₂—(C₁₋₆)alkyl, CONRₐR_{b}, and NRₐR_{b};
R₃ is COOH, CONRₐR_{b}, SO₃H, SO₂NRₐR_{b}, CONRₐSO₂R_{b}, 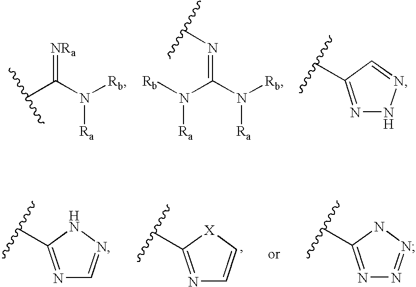
each Rₐ and R_{b} is independently selected from hydrogen, (C₁₋₆)alkyl, aryl, heterocycle, (C₁₋₆)alkyl-aryl, and (C₁₋₆)alkyl-heterocycle;
or a pharmaceutically acceptable salt thereof.

With respect to formula (I):

Suitably X Is O or NRₐ wherein Rₐ is hydrogen, (C₁₋₆)alkyl, or (C₁₋₆)alkyl-heterocycle.

Suitably B is phenyl, thiophene, furan, or pyridine.

Suitably R₃ Is COOH;

This invention also comprises novel compounds, which activate large conductance calcium activated potassium channels. This invention comprises compounds of formula (II): 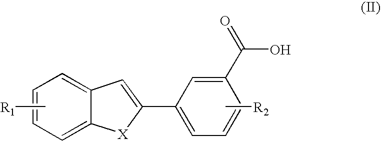
wherein:
R₁ is absent or represents up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, trifluoromethyl, cyano, CORₐ, CO₂Rₐ, SO₃H, (C₁₋₆)alkyl-CO₂—(C₁₋₆)alkyl, CONRₐR_{b}, and NRₐR_{b};
X is NRₐ, O, or S;
R₂ is absent or represents up to three substituents independently selected from (C₁₋₆)alkyl, (C₂₋₆)alkenyl, (C₃₋₆)cycloalkyl; aryl, (C₁₋₆)alkyl-aryl, heterocycle, (C₁₋₆)alkyl-heterocycle, ORₐ, SRₐ, hydroxy, halogen, nitro, cyano, CORₐ, SO₃H, (C₁₋₆)alkyl-CO₂—(C₁₋₆)alkyl, NRₐR_{b} and CO₂R_{c} wherein R_{c} is aryl, (C₁₋₆)-aryl, heterocycle, (C₁₋₆)alkyl-heterocycle, and (C₁₋₆)alkyl;
each Rₐ and R_{b} is independently selected from hydrogen, aryl, (C₁₋₆)alkyl-aryl, heterocycle, (C₁₋₆)alkyl-heterocycle, and (C₁₋₆)alkyl;
or a pharmaceutically acceptable salt thereof, provided that the compound is not 4-methoxy-3-(benzofuran-2-yl)-benzoic acid or 3-(5,6-dichloro-1H-indol-2-yl)-benzoic acid.

With respect to formula (II):

Suitably each R₁ is independently methyl, halo, trifluoromethyl, morpholinyl, NRₐR_{b}, or ORₐ wherein each Rₐ and R_{b} is independently hydrogen, (C₁₋₆)alkyl or piperizine.

Suitably X is O or NRₐ wherein Rₐ is hydrogen, (C₁₋₆)alkyl, or (C₁₋₆)alkyl-heterocycle. More suitably X is O or NRₐ wherein Rₐ is hydrogen, methyl, or 4-ethylmorpholinyl.

Suitably R₂ is halo, (C₁₋₆)alkyl, ORₐ, or NRₐR_{b} wherein each Rₐ and R_{b} is independently hydrogen or (C₁₋₆)alkyl.

Another aspect of this invention is a compound according to formula (III): 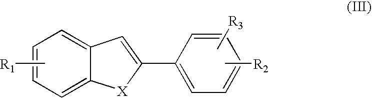
wherein:
R₁ is absent or represents up to three substituents Independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, trifluoromethyl, cyano, CORₐ, CO₂Rₐ, SO₃H, (C₁₋₆)alkyl-CO₂—(C₁₋₆)alkyl, CONRₐR_{b}, and NRₐR_{b};
X is NRₐ, O, or S;
R₂ is absent or represents up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, cyano, CORₐ, CO₂Rₐ, SO₃H, (C₁₋₆)alkyl-CO₂—(C₁₋₆)alkyl, and NRₐR_{b};
R₃ is SO₃H, SO₂NRₐR_{b}, CONRₐSO₂R_{b}, 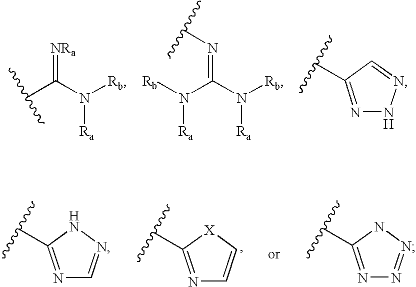
each Rₐ and R_{b} is independently selected from hydrogen, aryl, (C₁₋₆)-aryl, heterocycle, (C₁₋₆)alkyl-heterocycle, and (C₁₋₆)alkyl; or a pharmaceutically acceptable salt thereof.

Another aspect of this invention is a compound according to formula (IV): 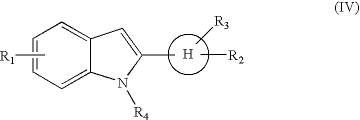
wherein:
R₁ is absent or represents up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, trifluoromethyl, cyano, CORₐ, CO₂Rₐ, SO₃H, (C₁₋₆)alkyl-CO₂—(C₁₋₆)alkyl, CONRₐR_{b}, and NRₐR_{b};
R₂ is absent or represents up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, cyano, CORₐ, CO₂Rₐ, SO₃H, (C₁₋₆)alkyl-CO₂—(C₁₋₆)alkyl, and NRₐR_{b};
R₃ is COOH, SO₃H, SO₂NRₐR_{b}, CONRₐSO₂R_{b}, 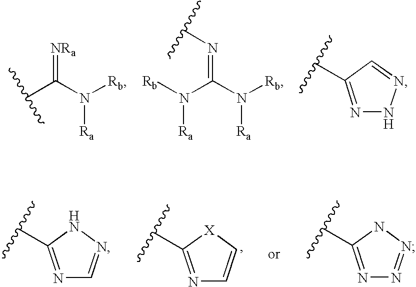
R₄ hydrogen, aryl, (C₁₋₆)-aryl, heterocycle, (C₁₋₆)alkyl-heterocycle, and (C₁₋₆)alkyl;
H is thiophene, furan, or pyridine.
each Rₐ and R_{b} is independently selected from hydrogen, aryl, (C₁₋₆)-aryl, heterocycle, (C₁₋₆)alkyl-heterocycle, and (C₁₋₆)alkyl; or a pharmaceutically acceptable salt thereof.

Representative of the novel compounds of this invention are the following:

5-(5,6-Dichloro-1H-Indol-2-yl)-furan-2-carboxylic acid;

3-(5,6-Dimethyl-1H-indol-2-yl)-benzoic acid;

3-(5,6-Dichloro-1H-indol-2-yl)-4-methoxy-benzoic acid;

5-(5,6-Dichloro-1H-indol-2-yl)-2-chloro-benzoic acid;

3-(5,6-Dichloro-1-methyl-indol-2-yl)-benzoic acid;

5-(5,6-Dimethyl-1H-indol-2-yl)-2-chloro-benzoic acid;

3-(5,6-Dimethyl-1H-indol-2-yl)-4-methoxy-benzoic acid;

3-(5-Chloro-benzofuran-2-yl)-benzoic acid;

3-(5,6-Dichloro-benzofuran-2-yl)-benzoic acid;

3-(Benzofuran-2-yl)-benzoic acid;

3-(5,6-Difluoro-benzofuran-2-yl)-benzoic acid;

5,6-Dichloro-2-[4-(1H-tetrazol-5-yl)-phenyl]-1H-indole; and

3-(1-Benzyl-5,6-dichloro-1H-indol-2-yl)-benzoic acid or

or a pharmaceutically acceptable salt thereof.

Representative compounds that treat or inhibit disorders associated with the activation of large conductance calcium activated potassium channels are the following:

3-(5,6-Dichloro-1H-indol-2-yl)-benzoic acid;

5-(5,6-Dichloro-1H-indol-2-yl)-furan-2-carboxylic acid;

3-(5,6-Dimethyl-1H-indol-2-yl)-benzoic acid;

3-(5,6-Dichloro-1H-indol-2-yl)-4-methoxy-benzoic acid;

5-(5,6-Dichloro-1H-indol-2-yl)-2-chloro-benzoic acid;

3-(5,6-Dichloro-1-methyl-indol-2-yl)-benzoic acid;

5-(5,6-Dimethyl-1H-indol-2-yl)-2-chloro-benzoic acid;

3-(5,6Dimethyl-1H-indol-2-yl)-4-methoxy-benzoic acid;

3-(5-Chloro-benzofuran-2-yl)-benzoic acid;

3-(5,6Dichloro-benzofuran-2-yl)-benzoic acid;

3-(Benzofuran-2-yl)-benzoic acid;

3-(5,6-Difluoro-benzofuran-2-yl)-benzoic acid; and

4-(5,6-Dichloro-1H-indol-2-yl)-benzoic acid; or a pharmaceutically acceptable salt thereof.

Also included in this invention are pharmaceutically acceptable addition salts and complexes of the compounds of this invention. In cases wherein the compounds of this invention may have one or more chiral centers, unless specified, this invention includes each unique nonracemic compound which may be synthesized and resolved by conventional techniques. In cases in which compounds have unsaturated carbon-carbon double bonds, both the cis (Z) and trans (E) isomers are within the scope of this invention. In cases wherein compounds may exist in tautomeric forms, such as keto-enol tautomers, such as 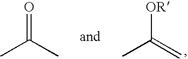
and each tautomeric form is contemplated as being included within this invention whether existing in equilibrium or locked in one form by appropriate substitution with R′.

Also included in this invention are prodrugs of the compounds of this invention. Prodrugs are considered to be any covalently bonded carriers which release the active parent drug according to formulae (II), (III), and (IV) in vivo.

The compounds of formulae (I) (II), (III), and (IV) and their pharmaceutically acceptable salts are BK channel activators. Activation of BK channels in bladder cells results in the relaxation of bladder smooth muscle tissue. Thus, the compounds of the instant invention are useful in the treatment of disorders involving excessive smooth muscle contraction of the urinary tract. These disorders include urinary incontinence, overactive bladder, pollakiuria, urge incontinence, diseases associated with detrusor instability, irritable bladder, cystitis, urethritis, and kidney stone ailments. Additionally, since the compounds of the instant invention activate BK channels, these compounds may also be useful in the treatment of other conditions or disease wherein the activation of BK channels ameliorates the condition. Such conditions or diseases are cerebral infarction, dimentia, Alzheimer's disease, Parkinson's disease, suprasacral spinalcord disease, central nervous system disorders, hypertension, stroke, angina, congestive heart failure, subarachnoid hemorrhage, irritable bowel syndrome, urethritis, kidney stone ailments, diverticuli or outflow obstruction, and brochial asthma, pain, inflammatory pain, neuropathic pain and chronic obstructive pulmonary disease (COPD).

Abbreviations and symbols commonly used in the peptide and chemical arts are used herein to describe the compounds of this invention.

Unless otherwise defined, the term (C₁₋₆)alkyl when used alone or when forming part of other groups (such as the ‘(C₁₋₆)alkyl-aryl’ group) includes substituted or unsubstituted, straight or branched chain alkyl groups containing 1 to 6 carbon atoms. Examples of (C₁₋₆)alkyl groups include methyl, ethyl, n-propyl, isopropyl, n-butyl, isobutyl, t-butyl, n-pentyl, isopentyl, neopentyl, and hexyl.

The term (C₂₋₆)alkenyl means a substituted or unsubstituted alkyl group of 2 to 6 carbon atoms, wherein one carbon-carbon single bond is replaced by a carbon-carbon double bond. Examples of (C₂₋₆)alkenyl include ethylene, 1-propene, 2-propene, 1-butene, 2-butene, and isobutene. Both cis and trans isomers are included.

The term (C₃₋₇)cycloalkyl refers to subsituted or unsubstituted carbocyclic ring system of three to seven carbon atoms, which may contain up to two unsaturated carbon-carbon bonds. Examples of (C₃₋₇)cycloalkyl include cyclopropyl, cyclobutyl, cyclopentyl, cyclopentenyl, cyclohexyl, cyclohexenyl, and cycloheptyl.

Unless otherwise defined, suitable substituents for any (C₁₋₆)alkyl, (C₂₋₆)alkenyl, and (C₃₋₇)cycloalkyl group, when used alone or when forming part of other groups (such as the ‘(C₁₋₆)alkyl-aryl’ group), includes up to five substituents, which may be on any carbon atom that results in a stable structure and is available by conventional synthetic techniques. Suitable substituents are halo, —OR′, —SR′, (C₁₋₆)alkylsulfonyl, (C₁₋₆)alkylsulfoxyl, —N(R′)₂, —CH₂N(R′)₂, nitro, cyano, —CO₂R′, —CON(R′)₂, —COR′, and —NR′C(O)R′, wherein each R′ is independently H or unsubstituted (C₁₋₆)alkyl.

Halo or halogen includes fluoro, chloro, bromo and iodo.

Ar or aryl, as applied herein, means phenyl or naphthyl, or phenyl or naphthyl substituted by one to three substituents, which may be on any carbon atom that results in a stable structure and is available by conventional synthetic techniques. Suitable substituents are halo, —OR′, —SR′, (C₁₋₆)alkylsulfonyl, (C₁₋₆)alkylsulfoxyl, —N(R′)₂, —CH₂N(R′)₂, nitro, cyano, —CO₂R′, —CON(R′)₂, —COR′, and —NR′C(O)R′, wherein each R′ is independently H or unsubstituted (C₁₋₆)alkyl.

The term ‘het’ or ‘heterocycle’ indicates a unsubstituted or substituted five or six membered monocyclic ring, or a nine or ten membered bicyclic ring containing one to four heteroatoms chosen from the group of nitrogen, oxygen, and sulfur, which is stable and available by conventional chemical synthesis. Illustrative heterocycles are benzofuran, benzimidazole, benzopyran, benzothiophene, benzothiazole, furan, imidazole, indoline, morpholine, piperidine, piperazine, pyrrole, pyrrolidine, tetrahydropyridine, pyridine, thiazole, oxazole, thiophene, quinoline, isoquinoline, pyrrolidine, pyridine, and piperizine. Unless otherwise defined, any heterocycle group contains up to three substitutents selected from the group of halo, —OR′, —SR′, (C₁₋₆)alkylsulfonyl, (C₁₋₆)alkylsulfoxyl, —N(R′)₂, —CH₂N(R′)₂, nitro, cyano, —CO₂R′, —CON(R′)₂, —COR′, and —NR′C(O)R′, wherein each R′ is independently H or unsubstituted (C₁₋₆)alkyl.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical, Bn refers to the benzyl radical, Me refers to methyl, Et refers to ethyl, Ac refers to acetyl, Alk refers to C₁₋₄alkyl, Nph refers to 1- or 2-naphthyl and cHex refers to cyclohexyl. Tet refers to 5-tetrazolyl.

Certain reagents are abbreviated herein. DCC refers to dicyclohexylcarbodiimide, DMAP refers to dimethylaminopyridine, DIEA refers to diisopropylethyl amine, EDC refers to 1-(3-dimethylaminopropyl)-3-ethylcarbodilmide, hydrochloride. HOBt refers to 1-hydroxybenzotriazole, THF refers to tetrahydrofuran, DIEA refers to diisopropylethylamine, DEAD refers to diethyl azodicarboxylate, PPh₃ refers to triphenylphosphine, DIAD refers to diisopropyl azodicarboxylate, DME refers to dimethoxyethane, DMF refers to dimethylformamide, NBS refers to N-bromosuccinimide, Pd/C refers to a palladium on carbon catalyst, PPA refers to polyphosphoric acid, DPPA refers to diphenylphosphoryl azide, BOP refers to benzotriazol-1-yloxy-tris(dimethyl-amino)phosphonium hexafluorophosphate, HF refers to hydrofluoric acid, TEA refers to triethylamine, TFA refers to trifluoroacetic acid, PCC refers to pyridinium chlorochromate.

Compounds of formulae II-IV are prepared by the general methods described in Schemes I-III.

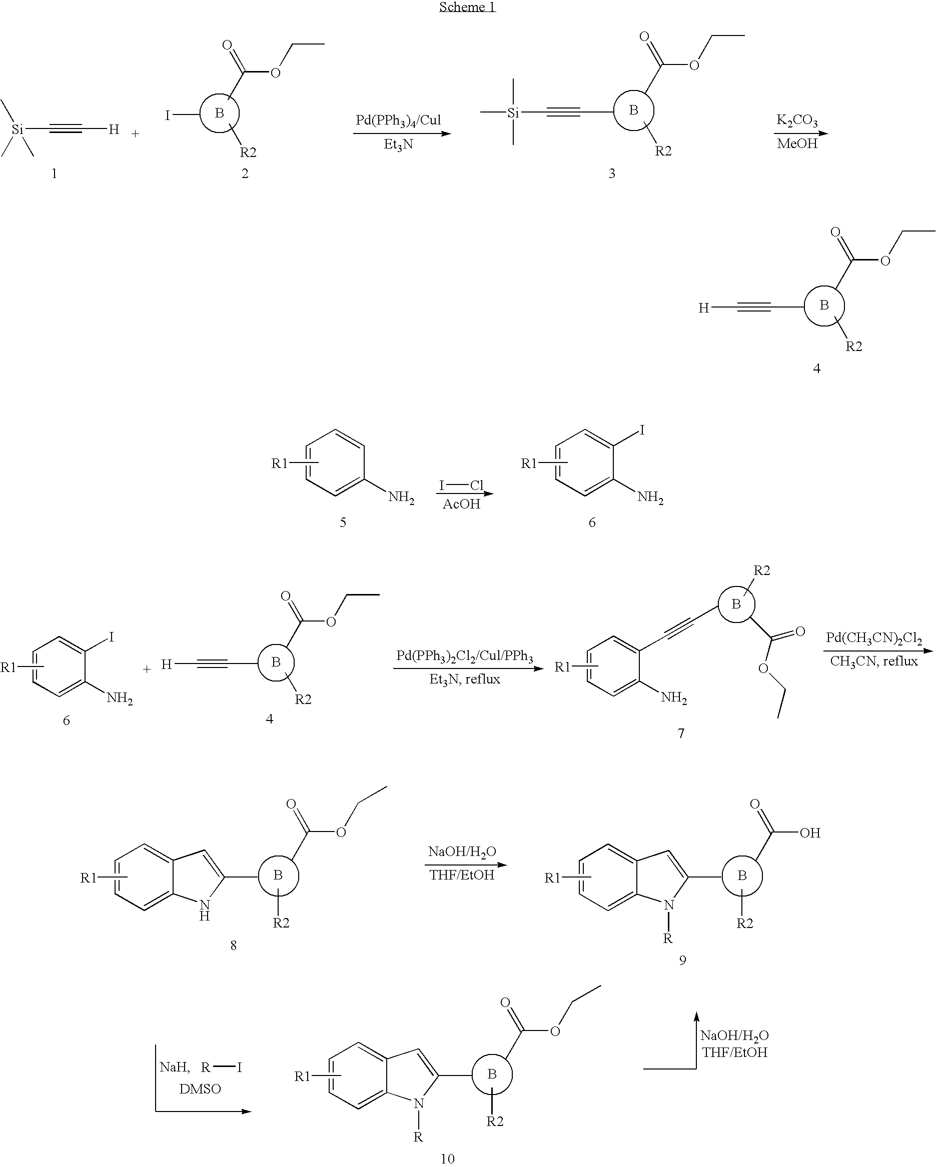

Scheme I represents a general scheme for the preparation of compounds according to Formula I wherein X is NRₐ. R₁ and R₂ are as defined above unless defined otherwise. R₃ is depicted as COOH; however, Scheme I may be used for preparing compounds wherein R₃ is any other defined group by substituting the appropriate starting materials. The starting materials and reagents for Scheme I are commercially available or are made from commercially available starting materials using methods known by those skilled in the art.

Trimethylsilylacetylene is reacted with an appropriate aryl- or heteroaryl-iodide (such as ethyl-2-iodo-benzoate and ethyl-5-bromo-furoate) in the presence of copper iodide, bis(triphenylphosphine)-dichloropalladium, and triethylamine to produce the desired trimethylsilyl-phenyl-actetylene, 3. The trimethylsilyl group is removed with potassium carbonate and methanol to produce 4. An aniline (such as 3,4-dichloro-aniline) is reacted with boron tribromide to produce the iodoaniline 6. The iodoaniline 6 is then reacted with the phenylacetylene, 4, in the presence of copper Iodide, bis(triphenylphosphine)-dichloropalladium, and triethylamine to afford the diphenylacetylene 7. The aniline 7 is heated in the presence of bis(acetonitrile)-dichloropalladium in acetonitrile to afford the cyclized product 8. The benzoate 8 is then hydrolyzed to the corresponding benzoic acid 9. Alternatively, benzoate 8 is alkylated using sodium hydride and an alkylhalide (such as methyl iodide) to afford N-alkylated product 10. The benzoate 10 is then hydrolyzed to the corresponding benzoic acid 9.

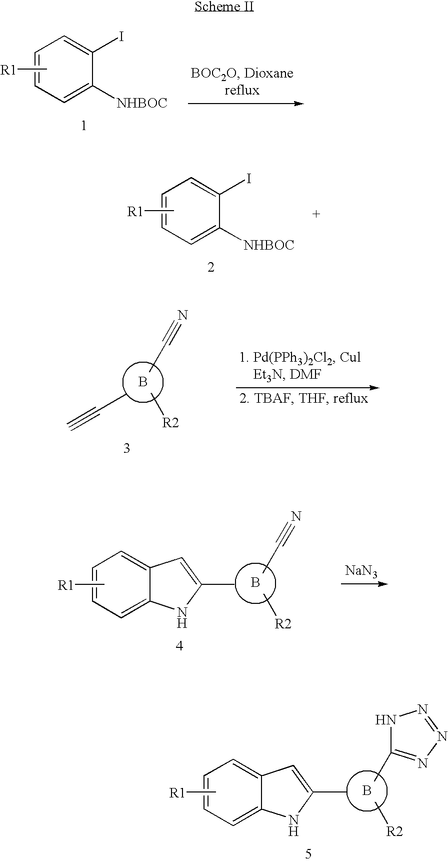

Scheme II represents an alternative scheme for the preparation of compounds according to Formula I wherein X is is NH and R₃ is tetrazolyl. R₁ and R₂ are as defined above unless defined otherwise. The starting materials and reagents for Scheme II are commercially available or are made from commercially available starting materials using methods known by those skilled in the art.

Iodo-aniline 1 is reacted with the BOC-anhydride in dioxane to produce the carbamate 2. Reaction of iodo-phenyl 2 with a substituted ethynyl-nitrile 3 in the presence of copper Iodide, bis(triphenylphosphine)-dichloropalladium, and triethylamine to afford a diphenylacetylene intermediate. The diphenylacetylene is then reacted with TBAF in refluxing THF to afford indole 4. The nitrile 4 is reacted with sodium azide in refluxing in 1-methyl-piperidin-2-one to afford the tetrazole 5.

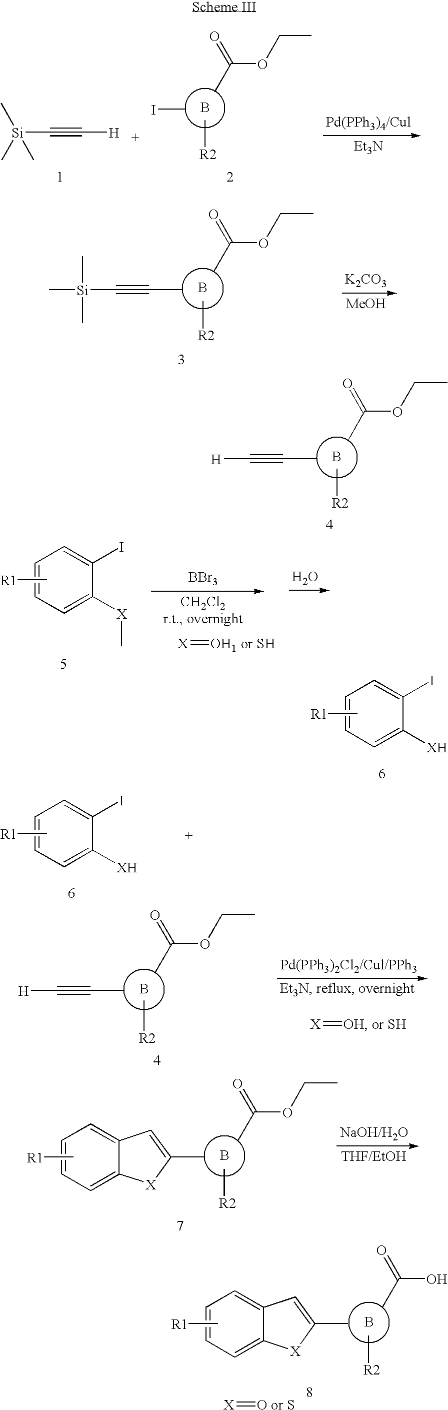

Scheme III represents a general scheme for the preparation of compounds according to Formula I wherein X is O or S. R₁ and R₂ are as defined above unless defined otherwise. R₃ is depicted as COOH; however, Scheme III may be used for preparing compounds wherein R₃ is any other defined group by substituting the appropriate starting materials. The starting materials and reagents for Scheme III are commercially available or are made from commercially available starting materials using methods known by those skilled in the art.

Trimethyl-acetylene is reacted with an appropriate aryl- or heteroaryl-iodide (such as ethyl-2-iodo-benzoate) in the presence of copper Iodide and bis(triphenylphosphine)-dichloropalladium to produce the desired trimethylsilyl-phenyl-acetetylene, 3. The trimethylsilyl group is removed with potassium carbonate and methanol to produce 4. An anisole (such as 4-chloro-anisole) may be reacted with boron tribromide to produce the iodophenol 6. An iodophenol (such as iodophenol, 2-iodo-4-chloro-phenol, or 2-iodo-4,5-dichloro-phenol) is then reacted with the phenyl-acetylene, 4, in the presence of copper iodide and bis(triphenylphosphine)dichloropalladium to afford the cyclized product 7. The ethyl benzoate is then hydrolyzed to the corresponding benzoic acid 8.

Acid addition salts of the compounds are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺and NH₄⁺ are specific examples of cations present in pharmaceutically acceptable salts.

This invention also provides a pharmaceutical composition which comprises a compound according to formulae (I), (II), (III), or (IV) and a pharmaceutically acceptable carrier. Accordingly, the compounds of formulae (I), (I), (III), and (IV) may be used in the manufacture of a medicament. Pharmaceutical compositions of the compounds of formulae (I), (II), (III), and (IV) prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in a emulsion or syrup for oral administration. Pharmaceutically acceptable solid or liquid carriers may be added to enhance or stabilize the composition, or to facilitate preparation of the composition. Solid carriers include starch, lactose, calcium sulfate dehydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

For topical administration, the compounds of this invention may be combined with diluents to take the form of ointments, gels, pastes, creams, powders or sprays. The compositions which are ointments, gels, pastes or creams contain diluents, for example, animal and vegetable fats, waxes, paraffins, starch, tragacanth, cellulose derivatives, polyethylene glycols, silicones, bentonites, silicic acid, talc and zinc oxide, or mixtures of these substances. The compositions which are powders or sprays contain diluents, for example, lactose, talc, silicic acid, aluminum hydroxide, calcium silicate and polyamide powder, or mixtures of these substances. Additionally, for topical ophthalmologic administration, the typical carriers are water, mixtures of water and water miscible solvents, such as lower alkanols or vegetable oils, and water-soluble non-toxic polymers, for example cellulose derivatives, such as methyl cellulose.

The compounds described herein are BK channel activators and are useful for treating conditions or diseases wherein the activation of BK channels would be desired or provide amelioration. For instance, these compounds are useful in the treatment of disorders associated with smooth muscle contraction and therefore, the instant compounds are useful in the treatment of disorders involving excessive smooth muscle contraction of the urinary tract. Thus, the instant compounds are useful in the treatment of urinary incontinence, overactive bladder, urge incontinence, diseases associated with detrusor instability, irritable bladder, pollakiuria, cystitis, urethritis, and kidney stone ailments. Because BK channels are also found on neuron cardiac and smooth muscle cells, the compounds of the instant invention are believed to have utility in the treatment of the following conditions or diseases: cerebral infarction, dimentia, Alzheimer's disease, Parkinson's disease, suprasacral spinalcord disease, central nervous system disorders, hypertension, stroke, angina, congestive heart failure, subarachnoid hemorrhage, irritable bowel syndrome, diverticuli or outflow obstruction, brochial asthma, pain, inflammatory pain, neuropathic pain and chronic obstructive pulmonary disease (COPD).

The compounds of this invention are administered to the patient, in a manner such that the concentration of drug is sufficient to treat urinary incontinence, or other such indications. The pharmaceutical composition containing the compound is administered at an oral dose of between about 10 mg to about 1000 mg, taken once or several times daily, in a manner consistent with the condition of the patient. Preferably, the oral dose would be about 50 mg to about 500 mg, although the dose may be varied depending upon the age, body weight and symptoms of the patient. For acute therapy, parenteral administration is preferred. An intravenous infusion of the compound of formula (I) in 5% dextrose in water or normal saline, or a similar formulation with suitable excipients, is most effective, although an intramuscular bolus injection is also useful. The precise level and method by which the compounds are administered is readily determined by one skilled in the art.

The compounds may be tested in one of several biological assays to determine the concentration of the compound which is required to have a given pharmaceutical effect.

Patch-Clamp Studies of BK Current in Freshly Isolated Bladder Smooth Muscle Cells.

Cell Isolation Bladders were removed from male Sprague-Dawley rats (250-400 g body weight) or male New Zealand White rabbits (2.5-3.5 kg body weight) killed by overdose with sodium pentobarbital. The urinary bladder was washed in cold, nominal Ca²⁺-free saline solution containing (in mM) 137 NaCl, 5 KH₂PO₄, 1 MgSO₄, 10 glucose, 5 HEPES, 8 taurine and 1 mg ml⁻¹ bovine serum albumin; pH=7.4. Small bundles of detrusor muscle were chopped into tiny pieces and incubated in the nominal Ca²⁺-free saline solution at room temperature for 30 minutes. The tissue pieces were then incubated at 37° C. in an enzyme solution made by adding 50 μM CaCl₂, 1.5 mg ml⁻¹ collagenase type II (Worthington Biochemical Corporation) and 1 mg ml⁻¹ protease XXIV (Sigma) to nominal Ca²⁺-free saline solution and bubbled with O₂. Single smooth muscle cells were harvested in the supernatant and the tissue pieces were re-incubated in fresh enzyme solution. Cell collection was repeated for 3 times. The greatest number of elongated cells were obtained around 90 and 120 minutes, respectively for rabbits and rats. The bladder smooth muscle cells were stored at 4° C. in a KB-medium composed of (in mM) 80 potassium glutamate, 20 K₂HPO₄, 20 KCl, 5 MgCl₂, 0.5 K₂EGTA, 2 Na₂ATP, 5 Na-pyruvate, 5 creatine, 20 taurine, 10 glycine, 10 glucose, and 5 HEPES. Cells were used for experiment within 8 hours.

BK current recording Cells were placed in a small experimental chamber constantly perfused with extracelluar solution (in mM): 140 NaCl, 4 KCl, 1 MgCl₂, 2 CaCl₂, 10 glucose, 10 HEPES; pH=7.4. The whole-cell voltage clamp technique was used for recording BK current The pipette solution was composed of (in mM) 140 KCl, 5 EGTA, 1 MgCl₂, 5 MgATP, 0.2 CaCl₂, 5 HEPES, pH=7.2. Drugs were dissolved in DMSO as 10 mM stocks and diluted to desired concentrations in extracellular solution. Cells were held at 0 mV and BK currents were recorded during 200-ms depolarizing voltage steps between 10 to 80 mV in 10-mV increments. Inter-pulse interval was 3-s. BK current amplitude was measured as the mean current during the last 30-ms of voltage steps and plotted against membrane voltage. The current/voltage relationships recorded in the absence and presence of various drugs were compared to determine the drug effects.

Compounds of the present invention display an increase in current greater than 5% control (basal response).

Effect of Compounds on KCl-Induced Contraction of Isolated Urinary Bladder Strips.

The urinary bladder was isolated from New Zealand White rabbits and cut into longitudinal strips (15 mm in length, 4 mm width). The mucosa was removed and the strips mounted in 15 ml vertical tissue baths, aerated with 95% O₂ and 5% CO₂, and bathed in a physiological salt solution of the following composition (mM): NaCl 118; KCl 4.7; NAHCO₃ 25; KH₂PO₄ 1.2; MgSO₄ 0.58; CaCl₂ 2.5 and glucose 11. The tissues were equilibrated for 1 h under 2 g resting tension and maintained at 37° C. The tissues were then precontracted by the addition of 15 mM KCl and after the response stabilized (approximately 20 min), test compounds were added cumulatively to the baths. Changes in tension were recorded using isometric force transducers connected to a PC based recording and analysis system and expressed as a percentage of relaxation produced by 0.1 mM papaverine.

A compound is considered to relax smooth muscle If the compound exhibits greater than 10% relaxation of smooth muscle at 10 μM compound concentration. Certain compounds of this invention show greater than 10% smooth musle relaxation.

The examples which follow are intended in no way to limit the scope of this invention, but are provided to illustrate how to make and use the compounds of this invention. Many other embodiments will be readily apparent to those skilled in the art.

### EXAMPLES AND EXPERIMENTAL

### General

Nuclear magnetic resonance spectra were recorded at 400 MHz using a Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (8) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kleseigel 60 (230-400 mesh) silica gel.

### Example 1

### Preparation of 3-(5,6-Dichloro-1H-indol-2-yl)-benzoic acid

### a) 2-Iodo-3,4-dichloro-aniline

3,4-Dichloroaniline (10.00 g, 61.73 mmol) was dissolved under argon in acetic acid (150 mL). ICI (15 g, 92.6 mmol) was dissolved in acetic acid (125 mL), and added slowly to the aniline solution over a period of one hour. After three hours, the reaction mixture was filtered, and the solids were washed with a small amount of acetic acid to give tan colored crystals. These crystals were triturated with water and filtered to give cream white solids (6.00 g). Additional solids had formed in the acetic acid filtrate. These were filtered, triturated with water to give cream colored solids, 2.09 g. Both crystalline batches were combined and recrystallized from warm cyclohexane (35 mL) to give white 4,5-dichloro-2-iodoaniline as crystalline solid, 6.25 g (35%), mp 79.5-80.5° C. TLC: ethyl acetate-hexane 1:9, single spot. LCMS 288 (M+H). NMR (d₆ DMSO), 7.74 (s, 1 H), 6.92 (s,1 H), 5.63 (s,2H).

### b) 3-(2-Amino-4,5-dichloro-phenylethynyl)-benzoic acid ethyl ester

To a stirring solution of 2-iodo-3,4-dichloro-aniline (1.2 g, 4.16 mmol) was added 3-ethynyl-benzoic acid ethyl ester (0.72 g, 4.14 mmol; Iijima, Toru; Endo, Yasuyuki, Tsuji, Motonori; Kawachi, Emico; Kagechika, Hiroyuki; Shudo, Koichi; Chem. Pharm. Bull. 1999, 47(3), 398-404) in triethylamine (20 mL) and THF (20 mL). To this solution was added copper iodide (7 mg, 0.037 mmol) and palladium bis(triphenylphosphine)dichloride. The mixture was stirred for 3.5 h at rt. The reaction mixture was concentrated and the crude product was dissolved in EtOAc. The EtOAc solution was washed with saturated, aqueous bicarbonate, H₂O, and brine. The EtOAc layer was dried over Na₂SO₄, filtered, and concentrated. The crude product was subjected to silica gel chromatography (15% EtOAc:Hexane) to afford the title compound, 0.66 g (47%). LCMS 334.2 (M+H).

### c) 4-(5,6-Dichloro-1H-indol-2-yl)-benzoic acid ethyl ester

To a stirring solution of the above aniline (0.33 g, 1.0 mmol) was added palladium bis(acetonitrile)dichloride in acetonitrile (25 mL). The reaction mixture was heated at 65° C. for 3 h. The mixture was cooled and filtered. The resulting crystalline product was washed in EtOH and dried under vacuum to provide the title compound as an off-white solid (0.2 g, 60%). LCMS 334.2 (M+H).

### d) 4-(5,6-Dichloro-1H-indol-2-yl)-benzoic acid

To a stirring solution of the above ester in EtOH (8 mL) and THF (8 mL) was added 1 N aq. NaOH (0.72 mL). The mixture was refluxed for 3.5 h, and concentrated. The residue was diluted with H₂O and acidified with HOAc. The resulting solid was dissolved in EtOAc, washed with H₂O, brine, dried over Na₂SO₄, and filtered. The EtOAc solution was concentrated to afford the title compound as a beige solid, 0.15 g (83%). LCMS 306.0 (M+H).

### Example 2

### Preparation of 5-(5,6-Dichloro-1H-indol-2-yl)-furan-2-carboxylic acid

The title compound was prepared in a similar manner to Example 1. LCMS 296.2 (M+).

### Example 3

### Preparation of 3-(5,6-Dimethyl-1H-indol-2-yl)-benzoic acid

The title compound was prepared in a similar manner to Example 1. LCMS 265.6 (M+).

### Example 4

### Preparation of 3-(5,6-Dichloro-1H-indol-2-yl)-4-methoxy-benzoic acid

The title compound was prepared in a similar manner to Example 1. LCMS 336.2 (M+).

### Example 5

### Preparation of 5-(5,6-Dichloro-1H-indol-2-yl)-2-chloro-benzoic acid

The title compound was prepared in a similar manner to Example 1. LCMS 340.4 (M+).

### Example 6

### Preparation of 3-(5,6-Dichloro-1-methyl-indol-2-yl)-benzoic acid

### a) 4-(5,6-Dichloro-1-Me-indol-2-yl)-benzoic acid ethyl ester

To a stirring solution of the ester from Example 1 Steps a-c, (100 mg, 0.3 mmol), in DMSO (2 mL), was added 60% NaH (16 mg, 0.4 mmol). The mixture was heated at 45° C. for 45 min, and then MeI (0.05 ml, 0.8 mmol) was added. The reaction mixture was stirred for an additional 30 min at rt. The residue was diluted with H₂O and extracted with EtOAc. The EtOAc layer was separated, and washed with H₂O, brine, dried over Na₂SO₄, filtered, and concentrated. To a flask containing the crude solid was added ether, and the heterogenous mixture was stirred for 10 min. The white solid was filtered and dried under vacuum to afford the title compound as a white solid, 0.07 g (67%). LCMS 348.0 (M+H).

### b) 4-(5,6-Dichloro-1-methyl-indol-2-yl)-benzoic acid

To a stirring solution of the above ester (70 mg, 0.2 mmol) in EtOH (8 mL) and THF (3 mL) was added 1 N aq. NaOH (0.2 mL). The mixture was refluxed for 3.5 h, and concentrated. The residue was diluted with H₂O and acidified with HOAc. The resulting solid was dissolved in EtOAc, washed with H₂O, brine, dried over Na₂SO₄, and filtered. The EtOAc solution was concentrated to afford the title compound as a white solid, 60 mg (90%). LCMS 320.2 (M+H).

### Example 7

### Preparation of 3-(5,6-Dimethyl-1H-indol-2-yl)-4-methoxy-benzoic acid

The title compound was prepared in a similar manner to Example 1. LCMS 296.2 (M+).

### Example 8

### Preparation of 5-(5,6-Dimethyl-1H-indol-2-yl)-2-chloro-benzoic acid

The title compound was prepared in a similar manner to Example 1. LCMS 300.2 (M+).

### Example 9

### Preparation of 3-(5-chloro-1-benzofuran-2-yl)benzoic acid

### a) Ethyl 3-[(trimethylsilyl)ethynyl]benzoate

A stirring solution of ethynyl(trimethyl)silane (8.68 g, 88.6 mmole) and ethyl 3-iodobenzoate (16.5 g, 59.8 mmole) in 90 ml of dry triethylamine was degassed, cooled down to 0° C. and treated with CuI (79 mg, 0.41 mmole) and Pd(PPh₃)₄ ( 1.0 g, 0.86 mmole). The resulting mixture was heated to reflux at 90° C. overnight before being concentrated in vacuo, then diluted with 250 ml of ether, and filtered. The filtrate was concentrated to yield 18.6 g of the title compound as a dark brown oil. MS(ES) m/e 247.2 [M+H]⁺.

### b) Ethyl 3-ethynylbenzoate

A solution of ethyl 3-[(trimethylsilyl)ethynyl]benzoate (8.6 g, 88 mmole) in 250 ml of methanol was treated with K₂CO₃ (23.9 g, 239 mmole). The resulting mixture was stirred at room temperature for 3 hours and then filtered, concentrated, diluted with 500 ml of ether and filtered again. The filtrate was collected and the solvent was removed in vacuo. Purification by flash silica gel column chromatography (Hexane:EtOAc=20:1) afforded the title compound as a pale green solid (9.68 g, 93% for a-b). MS(ES) m/e 175.2 [M+H]⁺.

### c) Ethyl 3-(5-chloro-1-benzofuran-2-yl)benzoate

A stirring solution of 4-chloro-2-iodoanisole (2.68 g, 10 mmole) in dry dichloromethane (60 ml) was treated with boron tribromide (15.0 ml, 1 M solution in dichloromethane) at room temperature. The reaction was run overnight before being quenched with 100 ml of water. The resulting mixture was extracted with two portions (250 ml) of dichloromethane, the organic layer was washed with brine, dried over sodium sulfate and concentrated in vacuo. Purification by flash silica gel column chromatography yielded the title compound as a solid (2.5 g, 100%).

A stirring solution of the above 4-chloro-2-iodophenol (0.75 g, 2.96 mmole), ethyl 3-ethynylbenzoate (0.566 g, 3.25 mmole) and triphenylphosphine (59 mg, 0.225 mmole) in 15 ml of dry triethylamine was degassed and treated with CuI (5.7 mg, 0.03 mmole) and Pd(PPh₃)₂Cl₂ (42 mg, 0.06 mmole). The resulting mixture was heated at 90° C. overnight, cooled to RT and concentrated in vacuo. Preparative HPLC (CH₃CN 60%-98% over 10 minutes) yielded the title product (0.463 g, 52%) as a white solid. MS(ES) m/e 301.2 (M+).

### d) 3-(5-chloro-1-benzofuran-2-yl)benzoic acid

A solution of ethyl 3-(5-chloro-1-benzofuran-2-yl)benzoate (0.440 g, 1.46 mmole) in 10 ml of ethanol and 10 ml of THF was treated with NaOH (2.0 ml 1 M solution in water). The resulting solution was heated at 55° C. for 3 hours and then cooled to RT. The organic solvents were removed in vacuo and the resulting material was diluted in 100 ml of water and washed two times (50 ml) with dichloromethane. The pH of the inorganic layer was adjusted to ˜4 with AcOH and the resulting mixture was extracted three times wiht EtOAc (300 ml). The organic layers were combined, washed with water, brine and dried over sodium sulfate. :The organic extracts were concentrated to yield the title compound as a yellow solid (0.39 g, 98%). MS(ES) m/e 273.2 (M+).

### Example 10

### Preparation of 3-(5,6-Dichloro-1-benzofuran-2-yl)benzoic acid

Following the procedure of Example 1 steps a-d, except 4,5-dichloro-2-iodophenol was used instead of 5-chloro-2-iodophenol in step c, the title compound was synthesized. MS(ES) m/e 307.2 (M+).

### Example 11

### Preparation of 3-(1-benzofuran-2-yl)benzoic acid

Following the procedure of Example 1 steps a-d, except 2-iodophenol was used instead of 5-chloro-2-iodophenol in step c, the title compound was synthesized. MS(ES) m/e 238.8 (M+).

### Example 12

### Preparation of 3-(5,6-Difluoro-1-benzofuran-2-yl)benzoic acid

Following the procedure of Example 1 steps a-d, except 2-bromo-4,5-difluorophenol was used instead of 5-chloro-2-iodophenol in step c, the title compound was synthesized. MS(ES) m/e 275.2 [M+H]⁺.

### Example 13

### Preparation of 4-(5,6-Dichloro-1H-indol-2-yl)-benzoic acid

The title compound was prepared in a similar manner to Example 1. LCMS 306.0 (M+H).

### Example 14

### Preparation of 5,6-Dichloro-2-[4-(1H-tetrazol-5-yl)-phenyl]-1H-indole

### a) [4,5-Dichloro-2-(4-cyano-phenylethynyl)-phenyl]-carbamic acid t-butyl ester

To a stirring solution of (4,5-dichloro-2-iodo-phenyl)-carbamic acid t-butyl ester (1.2 g, 0.48 mmol) (prepared by reacting 2-iodo-3,4-dichloro-aniline and BOC₂O) was added 4-ethynyl-benzonitrile (0.16 g, 1.24 mmol) in triethylamine (0.8 mL) and DMF (16 mL). To this solution was added copper iodide (47 mg, 0.25 mmol) and palladium bis(triphenylphosphine) dichloride (88 mg, 0.12 mmol). The mixture was stirred for 3.5 h at rt. The reaction mixture was concentrated and the crude product was dissolved in EtOAc. The EtOAc solution was washed with saturated, aqueous bicarbonate, H₂O, and brine. The EtOAc layer was dried over Na₂SO₄ and filtered. The EtOAc extracts were triturated with CH₃CN to afford afford a precipitate. The off-white solids were washed with EtOAc and dried under vacuum to provide the title compound, 0.32 g (67%). LCMS 387.0 (M+H).

### b) 4-(5,6-Dichloro-1H-indol-2-yl)-benzonitrile

To a stirring solution of [4,5-dichloro-2-(4-cyano-phenylethynyl)-phenyl]-carbamic acid t-butyl ester (0.32 g, 0.83 mmol) was added tetrabutylammonium fluoride (1.7 mL, 1.74 mmol—from a 1 N solution in THF) in THF (25 mL). The reaction mixture was heated at reflux for 2 h. The reaction mixture was cooled to RT, concentrated to one half volume, and diluted with EtOAc (25 mL). The EtOAc solution was washed with saturated aqueous brine, dried over Na₂SO₄, and filtered. The EtOAc extracts were triturated with CH₃CN to afford a precipitate. The solids were washed with EtOAc and dried under vacuum to provide the title compound, 0.16 g (68%). LCMS 287.2 (M+H).

### c) 5,6-Dichloro-2-[4-(1H-tetrazol-5-yl)-phenyl]-1H-indole

To a stirring solution 4-(5,6-dichloro-1H-indol-2-yl)-benzonitrile (0.14 g, 0.5 mmol) was added sodium azide (0.1 g, 1.5 mmol) and triethylamine (0.1 g, 0.75 mmol) in 1-methyl-piperidin-2-one (5 mL). The reaction mixture was heated at 120° C. for 12 h. The reaction mixture was cooled to RT and poured in H₂O. The aqueous mixture was extracted with EtOAc (150 mL). The EtOAc extracts were washed with H₂O, saturated aqueous brine, dried over Na₂SO₄, filtered, and concentrated to give a tan-colored solid. The solids were stirred in CH₃CN (5 mL) and filtered to afford the title compound, 0.13 g (79%). LCMS 330.0 (M+H).

### Example 15

### Preparation of 3-(1-Benzyl-5,6-dichloro-1H-indol-2-yl)-benzoic acid

### a) 3-(1-Benzyl-5,6-dichloro-1H-indol-2-yl)-benzoic acid ethyl ester

To a stirring solution of 3-(5,6-dichloro-1H-indol-2-yl)-benzoic acid ethyl ester (1.2 g, 0.48 mmol) (Steps 1(a)-(c)) was added benzyl bromide (120 uL, 1.0 mmol) and K₂CO₃ (0.2 g, 1.45 mmol) in acetone (25 mL). The mixture was heated at reflux for 10 h. The reaction mixture was concentrated and the crude product was dissolved in EtOAc. The EtOAc solution was washed with H₂O, saturated aqueous NaCl, dried over Na₂SO₄, filtered, and concentrated. The crude product was purified by silica gel chromatography (20% EtOAc-Hexane) to afford the title compound, 0.13 g (77%). LCMS 424.0 (M+H).

### b) 3-(1-Benzyl-5,6-dichloro-1H-indol-2-yl)-benzoic acid

To a stirring solution of 3-(1-benzyl-5,6-dichloro-1H-indol-2-yl)-benzoic acid ethyl ester (60 mg, 0.14 mmol) was added 1 N aqueous NaOH (0.25 mL, 0.25 mmol) in a 1:1 mixture of THF-EtOH (1 mL total volume). The reaction mixture was stirred for 10 h at RT. The reaction mixture was concentrated, the remaining white solid was suspended in H₂O (2 mL), and then acidified with glacial acetic acid. The acidic solution was extracted with EtOAc. The EtOAc extracts were washed with H₂O, saturated aqueous NaCl, dried over Na₂SO₄, filtered, and concentrated. Acetonitrile was added to the crude product and the heterogenous mixture was stirred for 1 h. The mixture was filtered and the resulting solid was dried under vacuum to provide the title compound, 38 mg (69%). LCMS 396.2 (M+H).

What is claimed is:

1. A method of treating or inhibiting disorders associated with the activation of large conductance calcium activated potassium channels, which comprises administering to a subject in need thereof an effective amount of a compound according to formula (I): 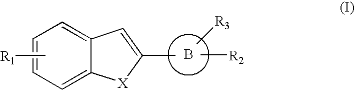
wherein:
R₁ is absent or represents up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, trifluoromethyl, cyano, CORₐ, CO₂Rₐ, SO₃H, (C₁₋₆)alkyl-CO₂—(C₁₋₆)alkyl, CONRₐR_{b}, and NRₐR_{b};
X is NRₐ, O, or S;
B is aryl or heterocycle;
R₂ is absent or represents up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, cyano, CORₐ, CO₂Rₐ, SO₃H, (C₁₋₆)alkyl-CO₂—(C₁₋₆)alkyl, CONRₐR_{b}, and NRₐR_{b};
R₃ is COOH, CONRₐR_{b}, SO₃H, SO₂NRₐR_{b}, CONRₐSO₂R_{b}, 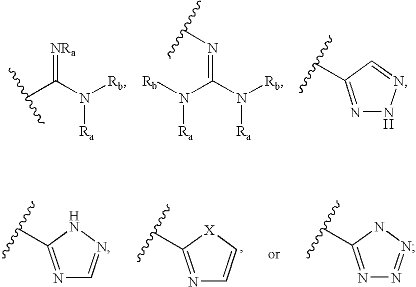
each Rₐ and R_{b} is independently selected from hydrogen, (C₁₋₆)alkyl, aryl, heterocycle, (C₁₋₆)alkyl-aryl, and (C₁₋₆)alkyl-heterocycle;
or a pharmaceutically acceptable salt thereof.

2. A method according to claim 1 of relaxing bladder smooth muscle tissue through the activation of large conductance calcium activated potassium channels.

3. A method according to claim 2 of treating urinary incontinence or overactive bladder.

4. A pharmaceutical composition which comprises a compound according to claim 1 and a pharmaceutically acceptable carrier.

5. A compound according to formula (II) 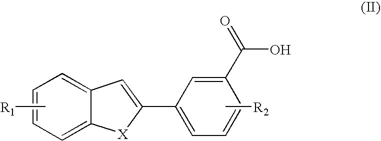
wherein:
R₁ is absent or represents up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, trifluoromethyl, cyano, CORₐ, CO₂Rₐ, SO₃H, (C₁₋₆)alkyl-CO₂—(C₁₋₆)alkyl, CONRₐR_{b}, and NRₐR_{b};
X is NRₐ, O, or S;
R₂ is absent or represents up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, cyano, CORₐ, SO₃H, (C₁₋₆)alkyl-CO₂—(C₁₋₆)alkyl, NRₐR_{b} and CO₂R_{c} wherein R_{c} is aryl, (C₁₋₆)-aryl, heterocycle, (C₁₋₆)alkyl-heterocycle, and (C₁₋₆)alkyl;
each Rₐ and R_{b} is independently selected from hydrogen, aryl, (C₁₋₆)-aryl, heterocycle, (C₁₋₆)alkyl-heterocycle, and (C₁₋₆)alkyl;
or a pharmaceutically acceptable salt thereof, provided that the compound is not 4-methoxy-3-(benzofuran-2-yl)-benzoic acid or 3-(5,6-dichloro-1H-indol-2-yl)-benzoic acid.

6. A compound according to formula (III) 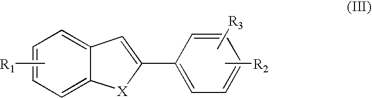
wherein:
R₁ is absent or represents up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, trifluromethyl, cyano, CORₐ, CO₂Rₐ, SO₃H, (C₁₋₆)alkyl-CO₂—(C₁₋₆)alkyl, CONRₐR_{b}, and NRₐR_{b};
X is NRₐ, O, or S;
R₂ is absent or represents up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, cyano, CORₐ, CO₂Rₐ, SO₃H, (C₁₋₆)alkyl-CO₂—(C₁₋₆)alkyl, and NRₐR_{b};
R₃ is SO₃H, SO₂NRₐR_{b}, CONRₐSO₂R_{b}, 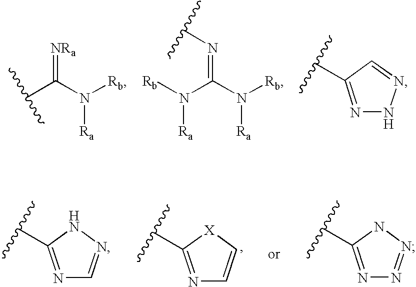
each Rₐ and R_{b} is independently selected from hydrogen, aryl, (C₁₋₆)-aryl, heterocycle, (C₁₋₆)alkyl-heterocycle, and (C₁₋₆)alkyl; or a pharmaceutically acceptable salt thereof.

7. A compound according to formula (IV) 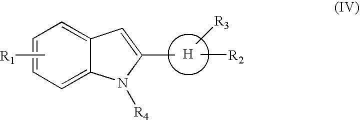
wherein:
R₁ is absent or represents up to three substituents independently selected from (C₁₋₆)alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, trilfuoromethyl, cyano, CORₐ, CO₂Rₐ, SO₃H, (C₁₋₆)alkyl-CO₂—(C₁₋₆)alkyl, CONRₐR_{b}, and NRₐR_{b};
R₂ is absent or represents up to three substituents independently selected from (C_{1- 6})alkyl, (C₂₋₆)alkenyl, (C₃₋₆)cycloalkyl, aryl, (C₁₋₆)alkyl-aryl, heterocycle, (C₁₋₆)alkyl-heterocycle, ORₐ, SRₐ, hydroxy, halogen, nitro, cyano, CORₐ, CO₂Rₐ, SO₃H, (C₁₋₆)alkyl-CO₂—(C₁₋₆)alkyl, and NRₐR_{b};
R₃ is COOH, SO₃H, SO₂NRₐR_{b}, CONRₐSO₂R_{b}, 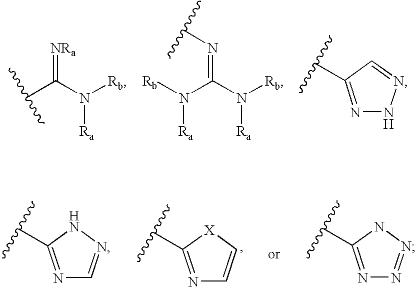
R₄ hydrogen, aryl, (C₁₋₆)-aryl, heterocycle, (C₁₋₆)alkyl-heterocycle, and (C₁₋₆)alkyl;
H is thiophene, furan, or pyridine.
each Rₐ and R_{b} is independently selected from hydrogen, aryl, (C₁₋₆)-aryl, heterocycle, (C₁₋₆)alkyl-heterocycle, and (C₁₋₆)alkyl; or a pharmaceutically acceptable salt thereof.

8. A compound which is:
5-(5,6-Dichloro-1H-indol-2-yl)-furan-2-carboxylic acid;
3-(5,6-Dimethyl-1H-indol-2-yl)-benzoic acid;
3-(5,6-Dichloro-1H-indol-2-yl)4-methoxy-benzoic acid;
5-(5,6-Dichloro-1H-indol-2-yl)-2-chloro-benzoic acid;
3-(5,6-Dichloro-1-methyl-indol-2-yl)-benzoic acid;
5-(5,6-Dimethyl-1H-indol-2-yl)-2-chloro-benzoic acid;
3-(5,6-Dimethyl-1H-indol-2-yl)-4-methoxy-benzoic acid;
3-(5-Chloro-benzofuran-2-yl)-benzoic acid;
3-(5,6-Dichloro-benzofuran-2-yl)-benzoic acid;
3-(Benzofuran-2-yl)-benzoic acid; or
3-(5,6-Difluoro-benzofuran-2-yl)-benzoic acid; or a pharmaceutically acceptable salt thereof.
